(19) United States
(12) Patent Application Publication
(10) Pub. No.: US 2019/0256928 A1
(43) Pub. Date: Aug. 22, 2019

(54) SYSTEMS AND METHODS FOR DETECTING DISSEMINATED OR CIRCULATING CELLS OR DNA

(71) Applicant: Fred Hutchinson Cancer Research Center, Seattle, WA (US)

(72) Inventor: Jason Bielas, Seattle, WA (US)

(73) Assignee: Fred Hutchinson Cancer Research Center, Seattle, WA (US)

(21) Appl. No.: 16/343,364

(22) PCT Filed: Oct. 20, 2017
(86) PCT No.: PCT/US17/57733

§ 371 Date: Apr. 18, 2019

Related U.S. Application Data
(60) Provisional application No. 62/410,789, filed on Oct. 20, 2016.

(51) Int. Cl. C12Q 1/6886; C12N 15/86

(52) U.S. Cl. CPC C12Q 1/6886; C12N 15/86; C12N 2740/15043; C12Q 2600/112; C12Q 2600/106

(57) ABSTRACT

Systems and methods for detecting disseminated or circulating cells or DNA are described. The systems and methods utilize genetic constructs including unique genetic sequences. The genetic constructs can be used to tag and track cancer cells. Digital polymerase chain reaction (dPCR), among other methods, can be used to quantitatively track the cells following administration.

### CROSS REFERENCE TO RELATED APPLICATION

This application claims priority to U.S. Provisional Patent Application No. 62/410,789 filed Oct. 20, 2016 which is incorporated herein by reference in its entirety as if fully set forth herein.

### FIELD OF THE DISCLOSURE

The current disclosure provides systems and methods for detecting disseminated or circulating cells or DNA. The systems and methods utilize genetic constructs including, for example, (i) repeats of unique genetic sequences separated by restriction enzyme sites; (ii) bar codes; and/or (iii) adapter sequences. The genetic constructs can be used to tag and track cells. Methods such as quantitative PCR (qPCR), digital PCR (dPCR) and/or next generation sequencing (NGS), among others, can be used to quantitatively track the cells following administration.

### BACKGROUND OF THE DISCLOSURE

The ability to detect disseminated or circulating cells would be beneficial in a number of settings. For example, the ability to detect and, optionally quantify, transplanted cells could further transplant medicine.

Measurement of disseminated tumor cells (DTCs), circulating tumor cells (CTCs), and circulating tumor DNA (ctDNA) is also a promising, non-invasive method for early disease detection, to diagnose and stage cancer, to assess response to therapy, and to evaluate progression and recurrence after surgery. However, to be practically useful, measurements must be able to detect 1 DTC, CTC or ctDNA molecule per millimeter of whole blood. While a technology termed CypherSeq met these high sensitivity and specificity requirements [Gregory, et al., Nucleic Acids Res, 2016. 44(3): p. e22], a single sample costs thousands of dollars to process. Thus, it remains cost-prohibitive to further investigate the utility of DTCs, CTCs, and ctDNA as markers for disease detection, predictors of metastatic recurrence, or to establish their respective kinetics during tumorigenesis, dissemination, and treatment response.

### SUMMARY OF THE DISCLOSURE

The present disclosure describes systems and methods to reliably and inexpensively detect disseminated cells, such as normal cells, transplanted cells, disseminated tumor cells (DTCs), circulating tumor cells (CTCs), and circulating tumor DNA (ctDNA). The disclosed systems and methods utilize unique genetic sequences. In particular embodiments, the unique genetic sequences are one or more of (i) repeats of unique genetic sequences separated by restriction enzyme sites (together collectively forming a genetic construct); and/or (ii) bar codes. The genetic construct can be used to tag and track cells (e.g., transplanted cells, DTCs, CTSs, and ctDNA). Methods such as quantitative PCR (qPCR), digital polymerase chain reaction (dPCR) and/or next generation sequencing (NGS), among others, can be used to quantitatively track the cells, for example, following administration.

The uniqueness of the genetic sequence allows tagging/detection of tagged cells following administration. In embodiments utilizing repeats of unique genetic sequences separated by restriction enzyme sites, the repetition of the unique sequence combined with interspersed restriction enzyme sites increases sensitivity of the systems and methods by reducing signal to noise ratios. In particular embodiments, accuracy and the quantitative nature of the assay is enhanced by amplifying the unique sequences using digital polymerase chain reaction (dPCR), and in particular embodiments, sample partition dPCR (spdPCR), one example of which is Droplet Digital™ PCR (ddPCRTM; Bio-Rad Laboratories, Hercules, Calif.).

In particular embodiments, the disclosed systems and methods can be used as a pre-clinical or clinical tool. In particular embodiments, the disclosed systems and methods can be used as a pre-clinical or clinical research tool. In particular embodiments, the disclosed systems and methods can aid in diagnosis and/or treatment of a subject in need thereof.

### BRIEF DESCRIPTION OF THE FIGURES

FIG. 1. A “spike-in” experiment illustrated that the random mutation capture (RMC) assay is sensitive enough to detect a single tumor cell amongst 1×10⁶ normal cells. This assay relies on detection of tumor mtDNA signatures amongst a background of normal mouse mtDNA genomes.

FIGS. 2A & 2B. Digital quantification of shed cancer cells. (1A) A schematic of an exemplary lentiviral tracking vector. In the depicted embodiment, ten unique, repetitive (e.g., identical) DNA sequences (e.g., an 80 bp fragment of C. elegans mitochondrial genome), with no significant homology to the mouse genome, are arranged in a head to tail fashion upstream of a luciferase GFP fusion reporter gene, which can be packaged and transfected into target cells. Each copy of the repetitive sequence can be flanked by a restriction site, which upon cleavage, allows for sequence enumeration by digital PCR. In the depicted embodiment, primers and TaqMan probes were designed to a fragment of C. elegans mitochondrial genome (unique molecular tag) and RPP30 (RNase P subunit 30) control locus detection by ddPCR. This control locus served as a reference gene, allowing measurement of the frequency of cancer cells in a tissue by genome-normalizing to a reference gene, which is present in all mouse cells at two copies per diploid genome. (1B) Primers and probes for ddPCR-based quantification of the molecular tag and RPP30 loci were combined with ddPCR mastermix and sample DNA, and then emulsified into a mixture of water-in-oil droplets (20,000 droplets per well), each droplet serving as an individual reaction chamber for PCR. These droplets are thermally cycled before being passed through a modified flow cytometer, where FAM (molecular tag) and VIC (RPP30) fluorescence are measured for each droplet. A FAM versus VIC plot allows gating of droplets with and without molecular tag and RPP30 loci, and then Poisson statistics are applied to these populations for accurate quantification.

FIG. 3. Exemplary repeated unique sequences (SEQ ID NOs: 9-34).

### DETAILED DESCRIPTION

The ability to detect disseminated or circulating cells would be beneficial in a number of settings. For example, the ability to detect and, optionally quantify, transplanted cells could further transplant medicine.

Measurement of disseminated tumor cells (DTCs), circulating tumor cells (CTCs), and circulating tumor DNA (ctDNA) is also a promising, non-invasive method for early disease detection, to diagnose and stage cancer, to assess response to therapy, and to evaluate progression and recurrence after surgery. However, the established frequency of dormant DTCs in patient specimens is 1 in 10⁶ normal cells. Thus, assays with exquisite sensitivity are required. To be practically useful, measurements must be able to detect, for example, 1 DTC, CTC or ctDNA molecule per millimeter of whole blood.

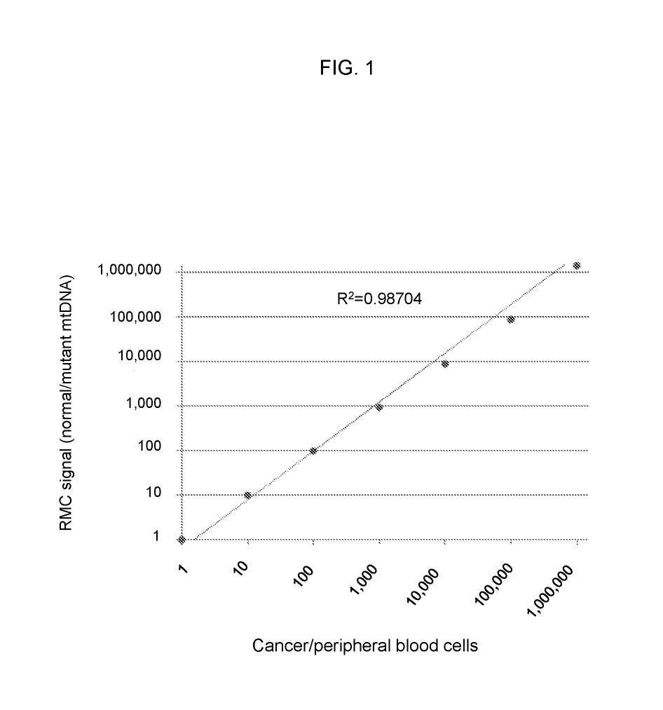

CypherSeq is an assay developed to address this problem. CypherSeq utilized the abundance of mitochondrial DNA (mtDNA) nucleotide polymorphisms that can uniquely identify human cell lines in a background of mouse cells. Exploiting these DNA marker sites in combination with an adapted and digitized Random Mutation Capture (RMC) assay [Bielas & Loeb, Nat Methods, 2005. 2(4): p. 285-90] accurately detected a single DTC diluted in stromal cells across six orders of magnitude (FIG. 1).

Although CypherSeq represented a major advance in the ability to track shed cancer cells in mice, it relied on counting unique human cells/DNA in mice, which necessitated that these studies be performed in mice with a compromised immune system. This caveat immediately precluded one from studying interactions between DTCs and adaptive immune cells for immunological or immunotherapeutic purposes. This fact, coupled with the ever-expanding evidence that the immune system plays numerous key roles in tumorigenesis, highlights the importance of using immunocompetent preclinical mouse models for cancer studies. Unfortunately, mouse cancer cells cannot be tracked in mouse tissues using CypherSeq (or any other known assay system), as there are very few potential DNA sequence differences between the implanted mouse cells and the mouse itself. Whereas rare implanted cell-specific sequences could be detected by employing CypherSeq [Gregory, et al., Nucleic Acids Res, 2016. 44(3): p. e22], this was fiscally inviable.

A different approach has been to utilize fluorescent markers in an attempt to measure and track DTCs, CTCs, and ctDNA. However, most traceable marker proteins, include the firefly luciferase (ffLuc) and/or jellyfish enhanced green fluorescent protein (eGFP), that are xenobiotic to mammals. Their expression alone induces various immune responses in immunocompetent animals, resulting in inconsistent activity, rejection of grafts, suppression of metastases, and/or failure to detect primary or metastatic lesions long-term. Thus, the effective use of xenobiotic reporters is restricted to either short-term studies, or fully immunocompromised animal models. The latter, of course, defeats the purpose of using syngeneic mouse tumor cells altogether.

To overcome these problems, a genetically engineered mouse that is immune-tolerant to both ffLuc and eGFP was developed to serve as a host for transplantation of labeled syngeneic tumors. By targeting minimal expression of a luciferase-GFP fusion reporter targeted to the anterior pituitary gland via a rat growth hormone promoter, the mouse tolerates these xenobiotic antigens but does not obscure ex vivo imaging of tumor growth at typical sites of metastasis. Whereas the use of pre-tolerized mice minimizes the immune response induced by xenobiotic reporters, it does not completely eliminate it [Day, et al., PLoS One, 2014. 9(11): p. e109956]. Moreover, the use of optically based reporters necessitates counting of cells essentially by eye, and the screened sample in many cases only represents a small cross section of target tissue. Thus, the results require extrapolation, and may not be representative of total metastatic burden. To get around this issue, some research labs use flow sorting [Lawson, et al., Nature, 2015. 526(7571): p. 131-5]. However, performing these tests in an exhaustive fashion on solid tissue requires digesting whole tissues and sorting every cell within said tissues. This is time consuming and costly.

The current disclosure provides a better approach that allows quantitative enumeration of cells (e.g., total tumor cell burden) within whole liquid and solid tissues in an accurate manner. This allows measurement of ctDNA to predict disease onset, drug response and recurrence and to measure efficacy of therapeutic regimens to eradicate dormant DTCs in all tissues of interest. Until the current disclosure, this ability was simply impossible.

The present disclosure describes systems and methods to reliably and inexpensively detect disseminated cells such as normal cells, transplanted cells, disseminated tumor cells (DTCs), circulating tumor cells (CTCs), and circulating tumor DNA (ctDNA). The disclosed systems and methods utilize genetic constructs including one or more of: (i) repeats of unique genetic sequences separated by restriction enzyme sites (together collectively forming a genetic construct); and/or (ii) bar codes. The genetic constructs can be used to tag and track cells (e.g., DTCs, CTSs, and ctDNA). The genetic constructs can also be used to tag and track implanted or administered cell types of a species within the same species (e.g., implanted mouse cells in mice and/or mouse tissues).

In particular embodiments, the uniqueness of a genetic sequence allows detection of the tagged cells. Repetition of the unique sequence combined with interspersed restriction enzyme sites increases sensitivity of the systems and methods by reducing signal to noise ratios. Accuracy and the quantitative nature of the systems and methods can be enhanced by amplifying the unique sequences using, for example, quantitative polymerase chain reaction (qPCR), digital PCR (dPCR), and/or other appropriate methods (e.g., NGS). In particular embodiments, sample partition dPCR (spdPCR), can be used, one example of which is Droplet Digital™ PCR (ddPCRTM) (Bio-Rad Laboratories, Hercules, Calif.).

In particular embodiments, the genetic constructs are recombinant genetic constructs. Recombinant genetic construct can refer to a genetic construct (e.g., a plasmid) that includes genetic material that would not be found in nature together (i.e., would not be present together in a naturally-occurring genome of an organism). A recombinant genetic construct can include: an artificial genetic sequence; an artificial genetic sequence and one or more sequences derived from one or more organism; and/or sequences derived from two or more organisms. In particular embodiments, the recombinant genetic construct includes a unique genetic sequence and a vector sequence that would not be found in nature together. In particular embodiments the recombinant genetic construct includes a unique genetic sequence and a restriction site that would not be found in nature together.

“Unique” means that the introduced genetic sequence can be readily distinguished from those genetic sequences naturally occurring in the organism into which the genetic sequence is introduced. In particular embodiments, genetic sequences can be readily distinguished because they do not naturally occur in the species in which the cells are implanted or administered. In particular embodiments, the unique sequences are readily distinguished because they have no homology with mouse sequences. In particular embodiments, the unique sequences are readily distinguished because they have no homology with human sequences. In particular embodiments, the unique sequences are readily distinguished because they have no homology with mouse sequences or with human sequences. In particular embodiments, the unique sequences are readily distinguishable because they are unique in, for example, 1-10 of 10-100 bases. Such sequences can be synthetically created sequences or can be sequences derived from a different organism (e.g., C. elegans, drosophila, C. intestinalis, Arabidopsis thaliana). In particular embodiments, the unique sequence is a sequence from C. elegans, drosophila, C. intestinalis (tunicate), S. purpuratus (sea urchin), Aquila chrysaetos (Golden Eagle), Arabidopsis thaliana, cow, zebrafish, stickleback fish, Saccharomyces cerevisiae, Rattus norvegicus (rat), Xenopus (frog), or Gallus gallus (chicken). In particular embodiments, the unique sequence can be a mitochondrial C. elegans sequence.

In particular embodiments, the genetic constructs (e.g., unique, repetitive, genetic sequences, bar codes and/or adapter sequences) are not transcribed and translated. This feature provides an important benefit because expressed non-native proteins are most often immunogenic. In other particular embodiments, however, a promoter can be placed upstream of a genetic construct so that the construct is transcribed and translated, and, for example, resulting RNA and/or protein can be detected.

In particular embodiments, a candidate sequence can be chosen as a unique sequence if it does not match any sequences present in the intended host recipient. A pairwise sequence alignment algorithm can be used to attempt to find any sequence matches between the candidate sequence and sequences present in the genome of its intended host recipient. An example of a pairwise sequence alignment algorithm that can be used is BLAST-like alignment tool (BLAT). An alignment tool such as BLAT can be used to search a genome (e.g., the mouse genome) for sequences that are identical to or are nearly identical to the candidate sequence.

Generally, and in particular embodiments, each unique genetic sequence can be at least 10 nucleotides in length and not more than 120 nucleotides in length. In particular embodiments, the unique genetic sequence is at least 18-36 nucleotides in length or at most 80-120 nucleotides in length.

The unique sequence is repeated in the genetic constructs described herein. In particular embodiments, the unique sequence can be a repeated unique sequence and can include at least 2 copies of the unique sequence; at least 3 copies of the unique sequence; at least 4 copies of the unique sequence; at least 5 copies of the unique sequence; at least 6 copies of the unique sequence; at least 7 copies of the unique sequence; at least 8 copies of the unique sequence; at least 9 copies of the unique sequence; at least 10 copies of the unique sequence; at least 11 copies of the unique sequence; at least 12 copies of the unique sequence; at least 13 copies of the unique sequence; at least 14 copies of the unique sequence; at least 15 copies of the unique sequence; at least 16 copies of the unique sequence; at least 17 copies of the unique sequence; at least 18 copies of the unique sequence; at least 19 copies of the unique sequence; or at least 20 copies of the unique sequence. In particular embodiments, the number of sequences is chosen to allow accurate quantification utilizing sample partition digital PCR (spdPCR). In particular embodiments, accurate means that the systems and methods can detect 1 cell in 100; 1 cell in 1000; 1 cell in 10,000; 1 cell in 100,000; or 1 cell in 1,000,000. Such accuracy can be assessed by performing spike-in experiments.

As will be understood by one of ordinary skill, complete repetitive identity between the unique sequences is not required. In particular embodiments, a degree of identity is required that allows amplification by a common primer sequence. This degree of identity may be 80% sequence identify; 81% sequence identify; 82% sequence identify; 83% sequence identify; 84% sequence identify; 85% sequence identify; 86% sequence identify; 87% sequence identify; 88% sequence identify; 89% sequence identify; 90% sequence identify; 91% sequence identify; 92% sequence identify; 93% sequence identify; 94% sequence identify; 95% sequence identify; 96% sequence identify; 97% sequence identify; 98% sequence identify; 99% sequence identify; or 100% sequence identify.

“% sequence identity” refers to a relationship between two or more sequences, as determined by comparing the sequences. In the art, “identity” also means the degree of sequence relatedness between sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine sequence identity are designed to give the best match between the sequences tested. Methods to determine sequence identity and similarity are codified in publicly available computer programs. Sequence alignments and percent identity calculations may be performed using the Megalign program of the LASERGENE bioinformatics computing suite (DNASTAR, Inc., Madison, Wis.). Within the context of this disclosure it will be understood that where sequence analysis software is used for analysis, the results of the analysis are based on the “default values” of the program referenced. “Default values” mean any set of values or parameters which originally load with the software when first initialized.

In particular embodiments, restriction enzyme sites separate repeat units of the unique genetic sequences. In particular embodiments, a restriction enzyme site is interspersed between each unique, repetitive, genetic sequence. Exemplary restriction enzyme sites include Taql (TOGA), Pacl (TTAATTAA), Ascl (GGCGCGCC), BamHl (GGATCC), Bglll (AGATCT), EcoRl (GAATCC) and Xhol (CTCGAG).

The unique, repetitive, genetic sequences with interspersed restriction enzyme sites (i.e., genetic constructs) can be introduced into cells using any appropriate vector. The vector should include all elements required for packaging, transduction, stable integration of the viral expression construct into genomic DNA, and expression of a reporter (e.g., the luciferase/eGFP optical fusion).

In particular embodiments, barcodes (e.g., double stranded bar codes) can be used. In particular embodiments, barcodes refer to DNA sequences that can utilized to identify the origin of a sample. In particular embodiments, these barcodes can be designed to be unique. In particular embodiments, DNA barcodes can include standardized short sequences of DNA (400-800 bp) characterized, in theory, for all species on the planet. Kress and Erickson, Proc. Natl. Acad. Sci. USA, 105(8): 2761-2762; Savolainen et al., Trans R Soc London Ser B. 2005; 360:1805-1811.

In particular embodiments, a unique sequence is inserted into a cell using a vector. A “vector” is a nucleic acid molecule that is capable of transporting another nucleic acid. Vectors may be, e.g., viruses, phage, a DNA vector, a RNA vector, a viral vector, a bacterial vector, a plasmid vector, a cosmid vector, and an artificial chromosome vector.

“Retroviruses” are a family of viruses having an RNA genome. In particular embodiments, a retroviral vector contains all of the cis-acting sequences necessary for the packaging and integration of the viral genome, i.e., (a) a long terminal repeat (LTR), or portions thereof, at each end of the vector; (b) primer binding sites for negative and positive strand DNA synthesis; and (c) a packaging signal, necessary for the incorporation of genomic RNA into virions. More detail regarding retroviral vectors can be found in Boesen, et al., 1994, Biotherapy 6:291-302; Clowes, et al., 1994, J. Clin. Invest. 93:644-651; Kiem, et al., 1994, Blood 83:1467-1473; Salmons and Gunzberg, 1993, Human Gene Therapy 4:129-141; Miller, et al., 1993, Meth. Enzymol. 217:581-599; and Grossman and Wilson, 1993, Curr. Opin. in Genetics and Devel. 3:110-114. In particular embodiments, a transgene (e.g., a repeated unique sequence) can be present between the LTRs of the retroviral vector, so that upon transduction of a cell with the lentivirus, the transgene becomes integrated into the host cell's genome.

“Gammaretroviruses” refer to a genus of the retroviridae family. Exemplary gammaretroviruses include mouse stem cell virus, murine leukemia virus, feline leukemia virus, feline sarcoma virus, and avian reticuloendotheliosis viruses.

Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. 66:2731-2739, 1992; Johann et al., J. Virol. 66:1635-1640, 1992; Sommerfelt et al., Virol. 176:58-59, 1990; Wilson et al., J. Virol. 63:2374-2378, 1989; Miller et al., J. Virol. 65:2220-2224, 1991; and PCT/US94/05700).

Particularly suitable are lentiviral vectors. “Lentivirus” refers to a genus of retroviruses that are capable of infecting dividing and non-dividing cells and typically produce high viral titers. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

In particular embodiments, other retroviral vectors can be used in the practice of the methods of the invention. These include, e.g., vectors based on human foamy virus (HFV) or other viruses in the Spumavirus genera.

Additional examples of viral vectors include those derived from adenoviruses (e.g., adenovirus 5 (Ad5), adenovirus 35 (Ad35), adenovirus 11 (Ad11), adenovirus 26 (Ad26), adenovirus 48 (Ad48) or adenovirus 50 (Ad50)), adeno-associated virus (AAV; see, e.g., U.S. Pat. No. 5,604,090; Kay et al., Nat. Genet. 24:257 (2000); Nakai et al., Blood 91:4600 (1998)), alphaviruses, cytomegaloviruses (CMV), flaviviruses, herpes viruses (e.g., herpes simplex), influenza viruses, papilloma viruses (e.g., human and bovine papilloma virus; see, e.g., U.S. Pat. No. 5,719,054), poxviruses, vaccinia viruses, etc. See Kozarsky and Wilson, 1993, Current Opinion in Genetics and Development 3:499-503, Rosenfeld, et al., 1991, Science 252:431-434; Rosenfeld, et al., 1992, Cell 68:143-155; Mastrangeli, et al., 1993, J. Clin. Invest. 91:225-234; Walsh, et al., 1993, Proc. Soc. Exp. Bioi. Med. 204:289-300; and Lundstrom, 1999, J. Recept. Signal Transduct. Res. 19: 673-686. Examples include modified vaccinia Ankara (MVA) and NYVAC, or strains derived therefrom. Other examples include avipox vectors, such as a fowlpox vectors (e.g., FP9) or canarypox vectors (e.g., ALVAC and strains derived therefrom).

In particular embodiments, the efficiency of integration, the size of the DNA sequence that can be integrated, and the number of copies of a DNA sequence that can be integrated into a genome can be improved by using transposons. Transposons or transposable elements include a short nucleic acid sequence with terminal repeat sequences upstream and downstream. Active transposons can encode enzymes that facilitate the excision and insertion of nucleic acid into a target DNA sequence.

A number of transposable elements have been described in the art that facilitate insertion of nucleic acids into the genome of vertebrates, including humans. Examples include sleeping beauty (e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol2 (e.g., derived from medaka fish); TcBuster (e.g., derived from the red flour beetle Tribolium castaneum) and spinON. CRISPR-Cas systems may also be used.

In particular embodiments, delivery of a unique sequence to a cell involves a nonviral gene editing system. Examples of nonviral gene editing systems include CRISPR-Cas, TALENS, MegaTAL, and zinc finger nucleases (ZFNs).

Particular embodiments utilize a CRISPR-Cas system. As indicated, Guide RNA can be used, for example, with gene-editing agents such as CRISPR-Cas systems. CRISPR-Cas systems include CRISPR repeats and a set of CRISPR-associated genes (Cas).

The CRISPR repeats (clustered regularly interspaced short palindromic repeats) include a cluster of short direct repeats separated by spacers of short variable sequences of similar size as the repeats. The repeats range in size from 24 to 48 base pairs and have some dyad symmetry which implies the formation of a secondary structure, such as a hairpin, although the repeats are not truly palindromic. The spacers, separating the repeats, match exactly the sequences from prokaryotic viruses, plasmids, and transposons. The Cas genes encode nucleases, helicases, RNA-binding proteins, and a polymerase that unwind and cut DNA. Cast, Cas2, and Cas9 are examples of Cas genes.

The source of CRISPR spacers indicate that CRISPR-Cas systems play a role in adaptive immunity in bacteria. There are at least three types of CRISPR-Cas immune system reactions, and Cast and Cas2 genes are involved in spacer acquisition in all three. Spacer acquisition, involving the capture and insertion of invading viral DNA into a CRISPR locus occurs in the first stage of adaptive immunity. More particularly, spacer acquisition begins with Cast and Cas2 recognizing invading DNA and cleaving a protospacer, which is ligated to the direct repeat adjacent to a leader sequence. Subsequently, single strand extension repairs take place and the direct repeat is duplicated.

The next stage of CRISPR-related adaptive immunity involves CRISPR RNA (crRNA) biogenesis, which occurs differently in each type of CRISPR-Cas system. In general, during this stage, the CRISPR transcript is cleaved by Cas genes to produce crRNAs. In the type I system, Cas6e/Cas6f cleaves the transcript. The type II system employs a transactivating (tracr) RNA to form a dsRNA, which is cleaved by Cas9 and RNase III. The type III system uses a Cas6 homolog for cleavage.

In the final stage of CRISPR-related adaptive immunity, processed crRNAs associate with Cas proteins to form interference complexes. In type I and type II systems, the Cas proteins interact with protospacer adjacent motifs (PAMs), which are short 3-5 bp DNA sequences, for degradation of invading DNA, while the type III systems do not require interaction with a PAM for degradation. In the type III-B system, the crRNA basepairs with the mRNA, instead of the targeted DNA, for degradation.

CRISPR-Cas systems thus function as an RNAi-like immune system in prokaryotes. The CRISPR-Cas technology has been exploited to inactivate genes in human cell lines and cells. As an example, the CRISPR-Cas9 system, which is based on the type II system, has been used as an agent for genome editing.

The type II system requires three components: Cas9, crRNA, and tracrRNA. The system can be simplified by combining tracrRNA and crRNA into a single synthetic single guide RNA (sg RNA).

At least three different Cas9 nucleases have been developed for genome editing. The first is the wild type Cas9 which introduces DSBs at a specific DNA site, resulting in the activation of DSB repair machinery. DSBs can be repaired by the NHEJ pathway or by homology-directed repair (HDR) pathway. The second is a mutant Cas9, known as the Cas9D10A, with only nickase activity, which means that it only cleaves one DNA strand and does not activate NHEJ. Thus, the DNA repairs proceed via the HDR pathway only. The third is a nuclease-deficient Cas9 (dCas9) which does not have cleavage activity but is able to bind DNA. Therefore, dCas9 is able to target specific sequences of a genome without cleavage. By fusing dCas9 with various effector domains, dCas9 can be used either as a gene silencing or activation tool.

Particular embodiments utilize transcription activator-like effector nucleases (TALENs) as gene editing agents. TALENs refer to fusion proteins including a transcription activator-like effector (TALE) DNA binding protein and a DNA cleavage domain. TALENs are used to edit genes and genomes by inducing double strand breaks (DSBs) in the DNA, which induce repair mechanisms in cells. Generally, two TALENs must bind and flank each side of the target DNA site for the DNA cleavage domain to dimerize and induce a DSB. The DSB is repaired in the cell by non-homologous end-joining (NHEJ) or by homologous recombination (HR) with an exogenous double-stranded donor DNA fragment.

As indicated, TALENs have been engineered to bind a target sequence of, for example, an endogenous genome, and cut DNA at the location of the target sequence. The TALEs of TALENs are DNA binding proteins secreted by Xanthomonas bacteria. The DNA binding domain of TALEs include a highly conserved 33 or 34 amino acid repeat, with divergent residues at the 12th and 13th positions of each repeat. These two positions, referred to as the Repeat Variable Diresidue (RVD), show a strong correlation with specific nucleotide recognition. Accordingly, targeting specificity can be improved by changing the amino acids in the RVD and incorporating nonconventional RVD amino acids.

Examples of DNA cleavage domains that can be used in TALEN fusions are wild-type and variant Fokl endonucleases. The Fokl domain functions as a dimer requiring two constructs with unique DNA binding domains for sites on the target sequence. The Fokl cleavage domain cleaves within a five or six base pair spacer sequence separating the two inverted half-sites.

Particular embodiments utilize MegaTALs as gene editing agents. MegaTALs have a single chain rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Particular embodiments utilize zinc finger nucleases (ZFNs) as gene editing agents. ZFNs are a class of site-specific nucleases engineered to bind and cleave DNA at specific positions. ZFNs are used to introduce DSBs at a specific site in a DNA sequence which enables the ZFNs to target unique sequences within a genome in a variety of different cells. Moreover, subsequent to double-stranded breakage, homologous recombination or non-homologous end joining takes place to repair the DSB, thus enabling genome editing.

ZFNs are synthesized by fusing a zinc finger DNA-binding domain to a DNA cleavage domain. The DNA-binding domain includes three to six zinc finger proteins which are transcription factors. The DNA cleavage domain includes the catalytic domain of, for example, Fokl endonuclease.

As previously stated, numerous methods can be used to amplify and/or sequence within the systems and methods disclosed herein. In particular embodiments, DNA segments are optionally amplified or increased by an amplification process, such as PCR, strand displacement amplification (SDA), and derivations thereof, to generate DNA segments, that is, amplification products. A DNA polymerase such as Taq or another thermostable polymerase can be used for amplification by PCR. See, e.g., Fakruddin et al., J Pharm Bioallied Sci. 5:245 (2013) for a review of amplification methods. In particular embodiments, amplification products can be used for sequencing.

Amplification may be performed with any suitable reagents (e.g. template nucleic acid (e.g. DNA or RNA)), primers, probes, buffers, replication catalyzing enzymes (e.g. DNA polymerase, RNA polymerase), nucleotides, salts (e.g. MgCl₂), etc. In particular embodiments, an amplification mixture includes any combination of at least one primer or primer pair, at least one probe, at least one replication enzyme (e.g., at least one polymerase), and deoxynucleotide (and/or nucleotide) triphosphates (dNTPs and/or NTPs), etc.

Exemplary mitochondrial C. elegans sequences that can be used in systems and methods disclosed herein include:
Forward: (GAGCGTCATTTATTGGGAAGA; SEQ ID NO: 1);
Reverse: (AATAAAGCTTGTGCTAATCCCAT; SEQ ID NO: 2);
Probe: (FAM-TCGTCTAGGGCCCACCAAGGTT (SEQ ID NO: 3)-TAMRA); and
Target: (GAGCGTCATTTATTGGGAAGAagacaaaatcgtctagggcccaccaaggttacatttATGGGATT AGCACAAGCTTTATT (SEQ ID NO: 4). For this exemplary embodiment, the target is mtDNA, positions 1838-1917.

An exemplary Drosophila melanogaster (“drosophila”) sequence that can be used in systems and methods disclosed herein includes: CAGATCGCATGGCCTGACGGAA GAGGCTCCGCCTCCCCCATCTCGCAAGGGCCTAAAGAAGCCCACATCGTCGGCGGTTG CCGCTTCACCCGCTTTAATG (SEQ ID NO: 5). For this exemplary target, the genomic position is dm6_dna 2L:873301-873400. Any unique fragment of this sequence as short as 18 base pairs in length can be used as a unique sequence.

An exemplary Golden eagle sequence that can be used in systems and methods disclosed herein includes: AGCAGCTTTGTAACTTCATCCATCATACTGATTTTATGGTATCCCTAAAG TCATTTGATACCAGTCTTTACGGTTTCCCCACTGACTACTCAGTAAC (SEQ ID NO: 6). For this exemplary target, the genomic position is KN265877v1:188145-188241. Any unique fragment of this sequence as short as 18 base pairs in length can be used as a unique sequence.

An exemplary S. purpuratus sequence that can be used in systems and methods disclosed herein includes: AGGGACAGTCGGTAAGCCGTAACTGTACGCTGAAATGACATCGG TTCGAACTTCCACCAACGAGCTACAAAGTGGCAAATGGACATAGAATACAGATTAA (SEQ ID NO: 7). For this exemplary target, the genomic position is Scaffold1:195992-196091. Any unique fragment of this sequence as short as 18 base pairs in length can be used as a unique sequence.

An exemplary C. intestinalis sequence that can be used in systems and methods disclosed herein includes: ATGCGGCAGCTAGTTGTTACGCAACATTATTAATGATCTATT CTATTGTGAGAATCGTGTTCTGTATCATCTACTGAGCGGTATACAAATGTTTCCCATT (SEQ ID NO: 8). For this exemplary target, the genomic position is chr1:5023254-5023353. Any unique fragment of this sequence as short as 18 base pairs in length can be used as a unique sequence.

In particular embodiments, adapter sequences can be used. Exemplary adapter sequences conform to primers used in particular embodiments. The Applied Biosystems SOLiD™ System sequencing platform for DNA uses truncated-TA adapters for capture of the DNA on the microarray and pre-capture amplification by PCR. See Protocol Version 2.1, Baylor College of Medicine, Human Genome Sequencing Center, “Preparation of SOLiD™ System Fragment Libraries for Targeted Resequencing using NimbleGen Microarrays or Solution Phase Sequence Capture.” In a further example, the Applied Biosystems SOLiD 4 System employs P1 and P2 adapters for sequencing and PCR primer recognition as set forth in the Library Preparation Guide (April 2010). Adapters which provide priming sequences for both amplification and sequencing of library fragments for use with the 454 Life Science GS20 sequencing system are described by F. Cheung, et al. BMC Genomics 2006, 7:272.

In particular embodiments, the amplification can be performed by sample partition dPCR (spdPCR). An example of sample partition dPCR is Droplet Digital PCR.

Droplet digital PCR (ddPCR) allows accurate quantification of tagged and tracked cells (e.g., Droplet Digital™ PCR (ddPCRTM) (Bio-Rad Laboratories, Hercules, Calif.)). ddPCR technology uses a combination of microfluidics and surfactant chemistry to divide PCR samples into water-in-oil droplets. Hindson et al., Anal. Chem. 83(22): 8604-8610 (2011). The droplets support PCR amplification of the target template molecules they contain and use reagents and workflows similar to those used for most standard Taqman probe-based assays.

Following PCR, each droplet is analyzed or read in a flow cytometer to determine the fraction of PCR-positive droplets in the original sample. These data are then analyzed using Poisson statistics to determine the target concentration in the original sample. See Bio-Rad Droplet Digital™ (ddPCR™) PCR Technology.

While ddPCR™ is a preferred spdPCR approach, other sample partition PCR methods based on the same underlying principles may also be used. These approaches are now described more generally.

Sample Partitioning. Numerous methods can be used to divide samples into discrete partitions (e.g., droplets). Exemplary partitioning methods and systems include use of one or more of emulsification, droplet actuation, microfluidics platforms, continuous-flow microfluidics, reagent immobilization, and combinations thereof. In particular embodiments, partitioning is performed to divide a sample into a sufficient number of partitions such that each partition contains one or zero nucleic acid molecules. In particular embodiments, the number and size of partitions is based on the concentration and volume of the bulk sample.

Methods and devices for partitioning a bulk volume into partitions by emulsification are described in Nakano et al. J Biotechnol 102, 117-124 (2003) and Margulies et al. Nature 437, 376-380 (2005). Systems and methods to generate “water-in-oil” droplets are described in U.S. Publication No. 2010/0173394. Microfluidics systems and methods to divide a bulk volume into partitions are described in U.S. Publication Nos. 2010/0236929; 2010/0311599; and 2010/0163412, and U.S. Pat. No. 7,851,184. Microfluidic systems and methods that generate monodisperse droplets are described in Kiss et al. Anal Chem. 80(23), 8975-8981 (2008). Further microfluidics systems and methods for manipulating and/or partitioning samples using channels, valves, pumps, etc. are described in U.S. Pat. No. 7,842,248. Continuous-flow microfluidics systems and methods are described in Kopp et al., Science, 280, 1046-1048 (1998).

Partitioning methods can be augmented with droplet manipulation techniques, including electrical (e.g., electrostatic actuation, dielectrophoresis), magnetic, thermal (e.g., thermal Marangoni effects, thermocapillary), mechanical (e.g., surface acoustic waves, micropumping, peristaltic), optical (e.g., opto-electrowetting, optical tweezers), and chemical means (e.g., chemical gradients). In particular embodiments, a droplet microactuator is supplemented with a microfluidics platform (e.g. continuous flow components).

Particular embodiments use a droplet microactuator. A droplet microactuator can be capable of effecting droplet manipulation and/or operations, such as dispensing, splitting, transporting, merging, mixing, agitating, and the like. Droplet operation structures and manipulation techniques are described in U.S. Publication Nos. 2006/0194331 and 2006/0254933 and U.S. Pat. Nos. 6,911,132; 6,773,566; and 6,565,727.

Amplification. The partitioned nucleic acids of a sample can be amplified by any suitable PCR methodology that can be practiced within spdPCR. Exemplary PCR types include allele-specific PCR, assembly PCR, asymmetric PCR, endpoint PCR, hot-start PCR, in situ PCR, intersequence-specific PCR, inverse PCR, linear after exponential PCR, ligation-mediated PCR, methylation-specific PCR, miniprimer PCR, multiplex ligation-dependent probe amplification, multiplex PCR, nested PCR, overlap-extension PCR, polymerase cycling assembly, qualitative PCR, quantitative PCR, real-time PCR, single-cell PCR, solid-phase PCR, thermal asymmetric interlaced PCR, touchdown PCR, universal fast walking PCR, etc. Ligase chain reaction (LCR) may also be used.

PCR may be performed with a thermostable polymerase, such as Taq DNA polymerase (e.g., wild-type enzyme, a Stoffel fragment, FastStart polymerase, etc.), Pfu DNA polymerase, S-Tbr polymerase, Tth polymerase, Vent polymerase, or a combination thereof, among others.

PCR and LCR are driven by thermal cycling. Alternative amplification reactions, which may be performed isothermally, can also be used. Exemplary isothermal techniques include branched-probe DNA assays, cascade-RCA, helicase-dependent amplification, loop-mediated isothermal amplification (LAMP), nucleic acid based amplification (NASBA), nicking enzyme amplification reaction (NEAR), PAN-AC, Q-beta replicase amplification, rolling circle replication (RCA), self-sustaining sequence replication, strand-displacement amplification, etc.

Amplification reagents can be added to a sample prior to partitioning, concurrently with partitioning and/or after partitioning has occurred. In particular embodiments, all partitions are subjected to amplification conditions (e.g. reagents and thermal cycling), but amplification only occurs in partitions containing target nucleic acids (e.g. nucleic acids containing sequences complementary to primers added to the sample). The template nucleic acid can be the limiting reagent in a partitioned amplification reaction. In particular embodiments, a partition contains one or zero target (e.g. template) nucleic acid molecules.

In particular embodiments, nucleic acid targets, primers, and/or probes are immobilized to a surface, for example, a substrate, plate, array, bead, particle, etc. Immobilization of one or more reagents provides (or assists in) one or more of: partitioning of reagents (e.g. target nucleic acids, primers, probes, etc.), controlling the number of reagents per partition, and/or controlling the ratio of one reagent to another in each partition. In particular embodiments, assay reagents and/or target nucleic acids are immobilized to a surface while retaining the capability to interact and/or react with other reagents (e.g. reagent dispensed from a microfluidic platform, a droplet microactuator, etc.). In particular embodiments, reagents are immobilized on a substrate and droplets or partitioned reagents are brought into contact with the immobilized reagents. Techniques for immobilization of nucleic acids and other reagents to surfaces are well understood by those of ordinary in the art. See, for example, U.S. Pat. No. 5,472,881 and Taira et al. Biotechnol. Bioeng. 89(7), 835-8 (2005).

Target Sequence Detection. Detection methods can be utilized to identify sample partitions containing amplified target(s) (i.e., unique sequences). Detection can be based on one or more characteristics of a sample partition such as a physical, chemical, luminescent, or electrical aspects, which correlate with amplification.

In particular embodiments, fluorescence detection methods are used to detect amplified target(s), and/or identification of sample partitions containing amplified target(s). Exemplary fluorescent detection reagents include TaqMan probes, SYBR Green fluorescent probes, molecular beacon probes, scorpion probes, and/or LightUp probes® (LightUp Technologies AB, Huddinge, Sweden). Additional detection reagents and methods are described in, for example, U.S. Pat. Nos. 5,945,283; 5,210,015; 5,538,848; and 5,863,736; PCT Publication WO 97/22719; and publications: Gibson et al., Genome Research, 6, 995-1001 (1996); Heid et al., Genome Research, 6, 986-994 (1996); Holland et al., Proc. Natl. Acad. Sci. USA 88, 7276-7280, (1991); Livak et al., Genome Research, 4, 357-362 (1995); Piatek et al., Nat. Biotechnol. 16, 359-63 (1998); Neri et al., Advances in Nucleic Acid and Protein Analysis, 3826, 117-125 (2000); Compton, Nature 350, 91-92 (1991); Thelwell et al., Nucleic Acids Research, 28, 3752-3761 (2000); Tyagi and Kramer, Nat. Biotechnol. 14, 303-308 (1996); Tyagi et al., Nat. Biotechnol. 16, 49-53 (1998); and Sohn et al., Proc. Natl. Acad. Sci. U.S.A. 97, 10687-10690 (2000).

In particular embodiments, detection reagents are included with amplification reagents added to the bulk or partitioned sample. In particular embodiments, amplification reagents also serve as detection reagents. In particular embodiments, detection reagents are added to partitions following amplification. In particular embodiments, measurements of the absolute copy number and the relative proportion of target nucleic acids in a sample (e.g. relative to other targets nucleic acids, relative to non-target nucleic acids, relative to total nucleic acids, etc.) can be measured based on the detection of sample partitions containing amplified targets.

In particular embodiments, following amplification, sample partitions containing amplified target(s) are sorted from sample partitions not containing amplified targets or from sample partitions containing other amplified target(s). In particular embodiments, sample partitions are sorted following amplification based on physical, chemical, and/or optical characteristics of the sample partition, the nucleic acids therein (e.g. concentration), and/or status of detection reagents. In particular embodiments, individual sample partitions are isolated for subsequent manipulation, processing, and/or analysis of the amplified target(s) therein. In particular embodiments, sample partitions containing similar characteristics (e.g. same fluorescent labels, similar nucleic acid concentrations, etc.) are grouped (e.g. into packets) for subsequent manipulation, processing, and/or analysis.

Particular embodiments, detecting and/or quantifying disseminated cells and/or disseminated genetic material can utilize NGS. In particular embodiments, DNA sequencing with commercially available NGS platforms may be conducted with the following steps. First, DNA sequencing libraries may be generated by clonal amplification by PCR in vitro. Second, the DNA may be sequenced by synthesis, such that the DNA sequence is determined by the addition of nucleotides to the complementary strand rather through chain-termination chemistry. Third, the spatially segregated, amplified DNA templates may be sequenced simultaneously in a massively parallel fashion without the requirement for a physical separation step. While these steps are followed in most NGS platforms, each utilizes a different strategy (see e.g., Anderson, M. W. and Schrijver, I., 2010, Genes, 1: 38-69.). Examples of NGS platforms include:

| | | | Read |
|---|---|---|---|
| | Template | | Length |
| Platform | Preparation | Chemistry | (basis) |
| Roche 454 | Clonal-emPCR | Pyrosequencing | 400 |
| GS FLX Titanium | Clonal-emPCR | Pyrosequencing | 400 |
| Illumina | Clonal Bridge | Reversible Dye | 35-100 |
| | Amplification | Terminator | |
| HiSeq 2000 | Clonal Bridge | Reversible Dye | 35-100 |
| | Amplification | Terminator | |
| Genom Analyzer IIX, | Clonal Bridge | Reversible Dye | 35-100 |
| IIE | Amplification | Terminator | |
| IScanSQ | Clonal Bridge | Reversible Dye | 35-75 |
| | Amplification | Terminator | |
| Life Technologies | Clonal-emPCR | Oligonucleotide | 35-50 |
| Solid 4 | | Probe Ligation | |
| Helicos Biosciences | Single Molecule | Reversible Dye | 35 |
| Heliscope | | Terminator | |
| Pacific Biosciences | Single Molecule | Phospholinked | 800-1000 |
| SMART | | Fluorescent | |
| | | Nucleotides | |

In particular embodiments, DNA segments can undergo an amplification as part of NGS sequencing. In embodiments where an amplification process was used to create a target-increased sample, this amplification would be a second amplification step. The second amplification can provide a stronger signal than if the second amplification was not performed.

In particular embodiments, the methods include quantifying and/or detecting an endogenous control. An endogenous control can refer to a sequence that has a known copy number in the cells of the subject, independent of the presence of the unique sequence. In particular embodiments, measuring the unique sequence and an endogenous control sequence can be useful for determining the copy number of the unique sequence. In particular embodiments, methods that include quantifying an endogenous control can be useful for determining the percentage of cells with the unique sequence in a sample (e.g., disseminated tumor cells). An exemplary method quantifying the copy number of a gene (e.g., a unique sequence) using an endogenous control can be found in Ma, L & Chung, W. Curr Protoc Hum Genet. 2014 Jan 21: 80: 7.21.1-7.21.8. Examples of endogenous controls are RNase P subunit 30 (RPP30) and TERT, both of which can be measured, for mouse or human samples, using commercially available reagents (e.g,. from THERMOFISHE®).

In particular embodiments, the methods include detecting an exogenous control. Exogenous control can refer to a DNA sequence that is “spiked” into a sample or a DNA extract from the sample. In particular embodiments, the exogenous control is spiked into the sample at a known quantity (e.g., known copy number), which can be useful, for example, to determine the absolute quantity of a gene sequence (e.g., a unique sequence).

In particular embodiments, methods of quantifying and/or detecting disseminated cells or disseminated genetic material can utilize an endogenous control or an exogenous control to determine the copy number of the unique sequence. In particular embodiments, copy number can also refer to relative copy number. Relative copy number can be, for example, a ratio of “copies of a unique sequence”: “copies of a control sequence”. Relative copy number can be useful, for example, for determining the percentage of cells that contain the unique sequence in a given sample, as a percentage of the total cells in the sample.

In particular embodiments, the methods disclosed herein can be used to detect disseminated cells and/or disseminated genetic material. In particular embodiments the disseminated cells contain a recombinant genetic construct and the detecting can be based on amplifying a unique sequence that is specifically associated with the disseminated cells. In particular embodiments, the disseminated genetic material can include at least a portion of a recombinant genetic construct, and the detecting can be based on amplifying a unique sequence that is part of the recombinant genetic construct.

In particular embodiments, the methods include detecting disseminated cells. Disseminated cells can refer to cells that have spread from one part of the body to another part of the body. Examples of disseminated cells can include cells that normally circulate through the body (e.g., immune cells), disseminated tumor cells, circulating tumor cells, and/or transplanted cells.

In particular embodiments the disseminated cells are disseminated unique cells. Disseminated unique cells can refer to disseminated cells that contain a unique sequence or a repeated unique sequence. For example, the disseminated unique cells can be cells transplanted in to a subject that does not naturally have cells that include the unique sequence. As another example, the disseminated unique cells can be a subset of a subject's cells that have been altered (e.g. with a lentiviral vector) to include a unique sequence that is not present in the unaltered cells of the subject.

In particular embodiments, the disseminated cells are transplanted cells. For example, the transplanted cells can be cancer cells transplanted into a research animal for researching cancer progression and/or metastasis. In particular embodiments, the transplanted cells contain a unique sequence or a repeated unique sequence that is not present in the subject's own cells.

In particular embodiments, the disseminated cells are disseminated tumor cells. Disseminated tumor cells can refer to tumor cells that have spread from one part of the body to another to another part of the body. For example, a disseminated tumor cell can be a metastatic tumor cell that originated as part of a primary tumor in a first location in the body (e.g., lung), and subsequently gave rise to a secondary tumor in a second location in the body (e.g., bone).

In particular embodiments the disseminated cells are circulating tumor cells. Circulating tumor cell can refer to a tumor cell that is circulating through the blood stream or the lymphatic system of a subject. Circulating tumor cells can give rise to secondary tumors, or cancer metastasis.

In particular embodiments, the methods include detecting disseminated genetic material. In particular embodiments, the disseminated genetic material is circulating tumor DNA (ctDNA), which can refer to tumor-derived, cell-free DNA fragments that circulate through the bloodstream or lymphatic system. The presence of ctDNA in the blood or lymphatic system can be indicative of cancer progression and/or metastasis.

In particular embodiments, detecting disseminated cells or ctDNA by quantifying a unique sequence associated with the cells or tumor cells that produce ctDNA can be useful for monitoring cancer progression and/or metastasis. For example, a research subject can be administered cancer cells that contain the unique sequence, and the progression or metastasis of the cancer cells can be monitored by obtaining samples from the research sample and measuring the unique sequence present in the sample. Increases in the amount of the unique sequence present in the samples derived from the research subject, over time, can indicate that the cancer is progressing. Administration of the cancer cells can include inducing a primary tumor in the research subject, and detecting the unique sequence in a sample such as a blood sample can be indicative of cancer metastasis.

In particular embodiments, the sample derived from the subject can be a blood sample, a lymph fluid sample, or any other tissue sample (such as a bone sample, a lung sample, or a brain sample). Blood and lymph samples can be particularly useful, for example, for measuring circulating cells, such as circulating tumor cells, and for measuring circulating tumor DNA.

### Exemplary Embodiments.

1. A recombinant genetic construct including five to twenty repeats of a sequence selected from SEQ ID NO: 4, SEQ ID NO: 5, SEQ ID NO: 6, SEQ ID NO: 7, or SEQ ID NO: 8 with a restriction enzyme site selected from Taql, Pacl, Ascl, BamHl, Bglll, EcoRl or Xhol interspersed between each repeat.
2. A recombinant genetic construct of embodiment 1 including a repeating subunit including SEQ ID NO: 9, SEQ ID NO: 10, SEQ ID NO: 11, SEQ ID NO: 12, SEQ ID NO: 30, SEQ ID NO: 31, SEQ ID NO: 32, or SEQ ID NO: 33, wherein the repeating subunit is repeated five to twenty times.
3. A recombinant genetic construct of embodiment 1 or 2 further including a viral vector.
4. A recombinant genetic construct of embodiment 3 wherein the viral vector includes a retroviral vector.
5. A recombinant genetic construct including at least two repeats of a genetic sequence that (i) is 18-120 base pairs in length; (ii) is not present in the mouse genome; and (iii) is not present in the human genome; wherein the repeats in the genetic sequence are separated by a restriction enzyme site.
6. A recombinant genetic construct of embodiment 5 wherein the repeats of the unique genetic sequence are identical.
7. A recombinant genetic construct of embodiment 5 or 6 wherein each of the repeats of the unique genetic sequence can be amplified by the same primer sequence.
8. A recombinant genetic construct of embodiment 5 or 7 wherein each of the repeats of the unique genetic sequence share at least 95% sequence identity.
9. A recombinant genetic construct of any of embodiments 5-8 wherein the unique genetic sequence is artificially created, derived from an animal, or derived from a plant.
10. A recombinant genetic construct of embodiment 9 wherein the unique genetic sequence is derived from C. elegans or drosophila.
11. A recombinant genetic construct of any of embodiments 5-10 wherein the restriction enzyme site includes one or more of Taql, Pacl, Ascl, BamHl, Bglll, EcoRl or Xhol.
12. A recombinant genetic construct of any of embodiments 5-11 including a repeating subunit comprising SEQ ID NO: 9, SEQ ID NO: 10, SEQ ID NO: 11, SEQ ID NO: 12, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 SEQ ID NO: 28, SEQ ID NO: 29, SEQ ID NO: 30, SEQ ID NO: 31, SEQ ID NO: 32, or SEQ ID NO: 33, wherein the repeating subunit is repeated five to twenty times.
13. A cell including a recombinant genetic construct of any of embodiments 1-12.
14. A cell of embodiment 13 wherein the cell is a transplanted cell.
15. A cell of embodiment 13 or 14 wherein the cell is a cancer cell.
16. A cell of embodiment 15 wherein the cancer cell is a mammalian cancer cell.
17. A cancer cell of embodiment 16 wherein the cancer cell is a mouse cancer cell or a human cancer cell.
18. A cancer cell of any of embodiments 15-17 wherein the cancer cell is an adrenal cancer cell, a bladder cancer cell, a blood cancer cell, a bone cancer cell, a brain cancer cell, a breast cancer cell, a carcinoma cell, a cervical cancer cell, a colon cancer cell, a colorectal cancer cell, a corpus uterine cancer cell, an ear, nose and throat (ENT) cancer cell, an endometrial cancer cell, an esophageal cancer cell, a gastrointestinal cancer cell, a head and neck cancer cell, a Hodgkin's disease cancer cell, an intestinal cancer cell, a kidney cancer cell, a larynx cancer cell, a leukemia cancer cell, a liver cancer cell, a lymph node cancer cell, a lymphoma cancer cell, a lung cancer cell, a melanoma cancer cell, a mesothelioma cancer cell, a myeloma cancer cell, a nasopharynx cancer cell, a neuroblastoma cancer cell, a non-Hodgkin's lymphoma cancer cell, an oral cancer cell, an ovarian cancer cell, a pancreatic cancer cell, a penile cancer cell, a pharynx cancer cell, prostate cancer cell, a rectal cancer cell, a sarcoma cancer cell, a seminoma cancer cell, a skin cancer cell, a stomach cancer cell, a teratoma cancer cell, a testicular cancer cell, a thyroid cancer cell, a uterine cancer cell, a vaginal cancer cell, a vascular tumor cancer cell, and/or a cancer cell from a metastasis thereof.
19. A method of quantitatively detecting disseminated cells in a subject including:

obtaining a sample derived from the subject;

detecting the presence of a unique genetic sequence and a control sequence in the sample by performing quantitative digital polymerase chain reaction (qPCR), digital PCR (dPCR) and/or next generation sequencing (NGS) on the sample; and

determining the copy number of the unique genetic sequence,

wherein the subject was administered a recombinant genetic construct of any of embodiments 1-12 and/or a cell including a recombinant genetic construct of any of embodiments 1-12.
20. A method of embodiment 19 wherein the detected disseminated cells are transplanted cells, disseminated tumor cells (DTCs), or circulating tumor cells (CTCs).
21. A method of embodiment 19 or 20 wherein the dPCR includes sample partition dPCR.
22. A method of embodiment 19 or 20 wherein the dPCR includes droplet dPCR.
23. A method of any of embodiments 19-22 wherein the subject is a mammal. 24. A method of any of embodiments 19-23 wherein the subject is a research animal.
25. A method of embodiment 24 wherein the research animal is a mouse, rat, monkey, pig, or dog.
26. A method of quantifying circulating tumor DNA in a subject including, obtaining a sample derived from the subject, detecting the presence of a unique sequence in the sample by performing quantitative digital polymerase chain reaction (qPCR), digital PCR (dPCR) and/or next generation sequencing (NGS) on the sample, and determining the copy number of the unique sequence in the sample, wherein the subject was administered a recombinant genetic construct of any of embodiments 1-12 or and/or a cell including a recombinant genetic construct of any of embodiments 1-12, and wherein the unique sequence is specifically associated with tumor cells in the subject, thereby quantifying circulating tumor DNA in the subject.
27. A method of embodiment 26 wherein the dPCR includes sample partition dPCR.
28. A method of embodiment 26 wherein the dPCR includes droplet dPCR.
29. A method of any of embodiments 26-28 wherein the subject is a mammal.
30. A method of any of embodiments 26-29 wherein the subject is a research animal.
31. A method of embodiment 30 wherein the research animal is a mouse, rat, monkey, pig, or dog.
32. A method of monitoring cancer progression or metastasis in a research subject including;

gene editing a cancer cell by inserting a repeated unique sequence into a human or mouse cell, wherein the unique sequence is (i) 18-120 base pairs in length, (ii) is not present in the mouse genome nor the human genome, and (iii) is repeated 5 to 20 times with restriction enzyme sites interspersed between each repeat;

administering the gene edited cancer cell to the research subject; and

monitoring the quantity of gene-edited cancer cells in the research subject by obtaining a sample derived from the subject; performing quantitative digital polymerase chain reaction (qPCR), digital PCR (dPCR) and/or next generation sequencing (NGS) on the sample; and determining the copy number of the repeated unique sequence in the sample,

thereby monitoring cancer progression or metastasis in the research subject.
33. A method of embodiment 32 wherein the gene-edited cancer cells are administered to induce a primary tumor in the research subject and the sample includes a blood sample.
34. A method of evaluating a cancer treatment in a research subject including;

gene editing a cancer cell by inserting a repeated unique sequence into a human or mouse cell, wherein the unique sequence is (i) 18-120 base pairs in length, (ii) is not present in the mouse genome nor the human genome, and (iii) is repeated 5 to 20 times with restriction enzyme sites interspersed between each repeat;

administering the gene edited cancer cell to the research subject;

administering a cancer treatment to the subject; and

monitoring the quantity of gene-edited cancer cells in the research subject by obtaining a sample derived from the subject; performing quantitative digital polymerase chain reaction (qPCR), digital PCR (dPCR) and/or next generation sequencing (NGS) on the sample; and determining the copy number of the repeated unique sequence in the sample,

thereby evaluating the cancer treatment in the subject.
35. A method of producing a research model for cancer progression or monitoring including gene editing a cancer cell by inserting a repeated unique sequence into a human or mouse cell, wherein the unique sequence is (i) 18-120 base pairs and length, (ii) is not present in the mouse genome nor the human genome, and (iii) is repeated 5 to 20 times with restriction enzyme sites between each repeat; and

inserting the gene edited cell into a research subject,

thereby producing a research model for cancer progression or monitoring.

### EXAMPLE 1

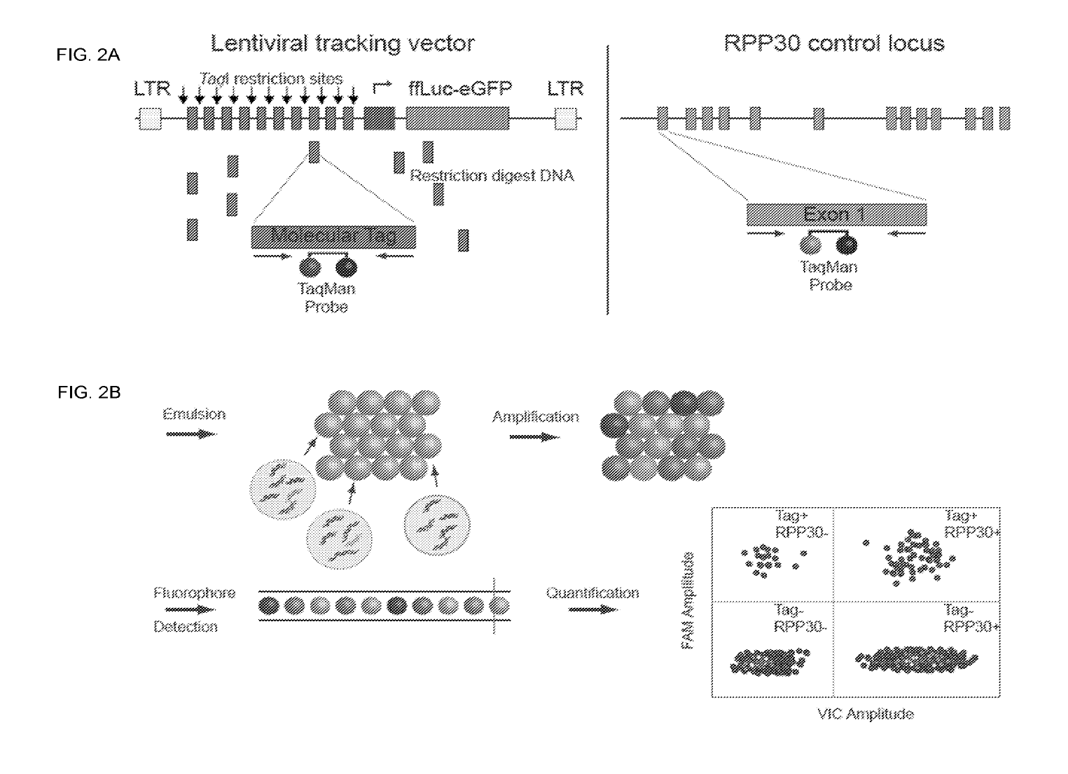

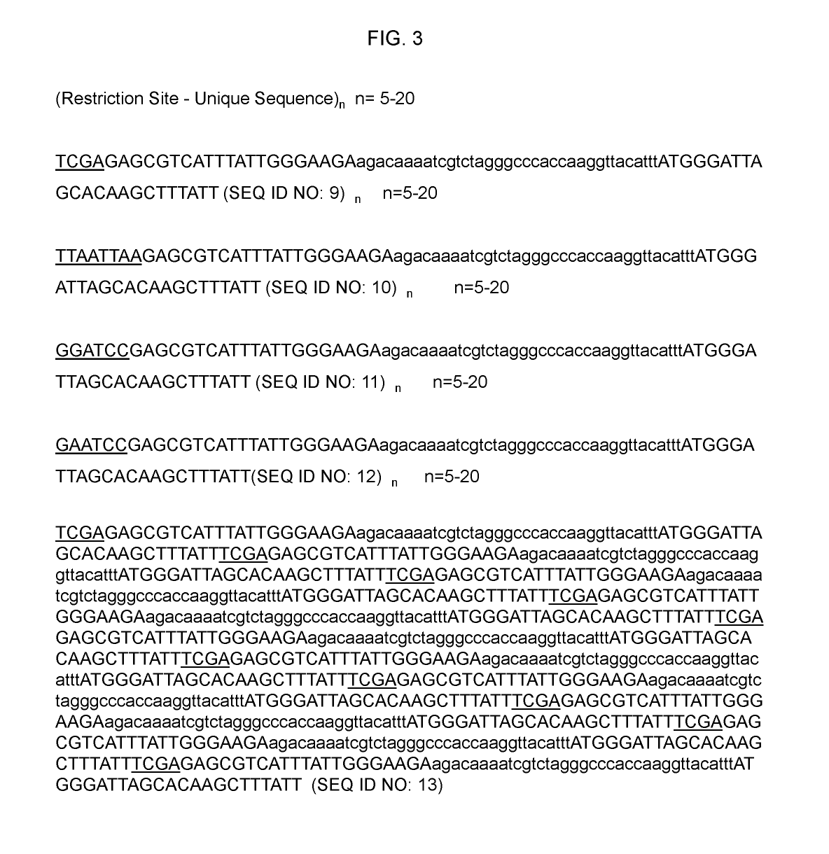

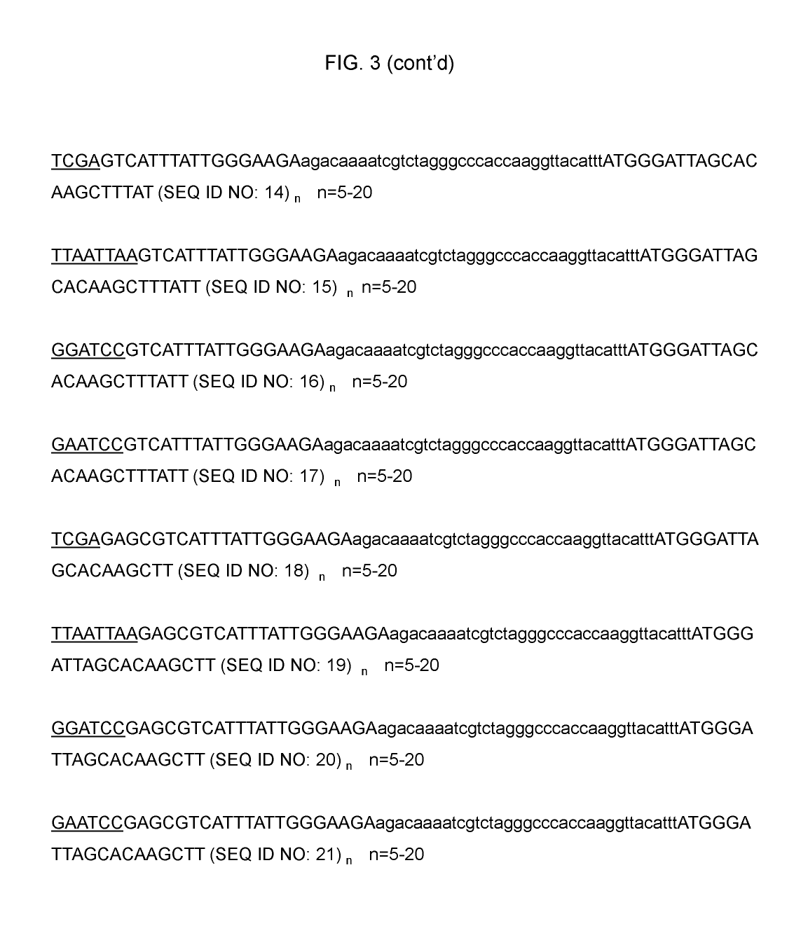

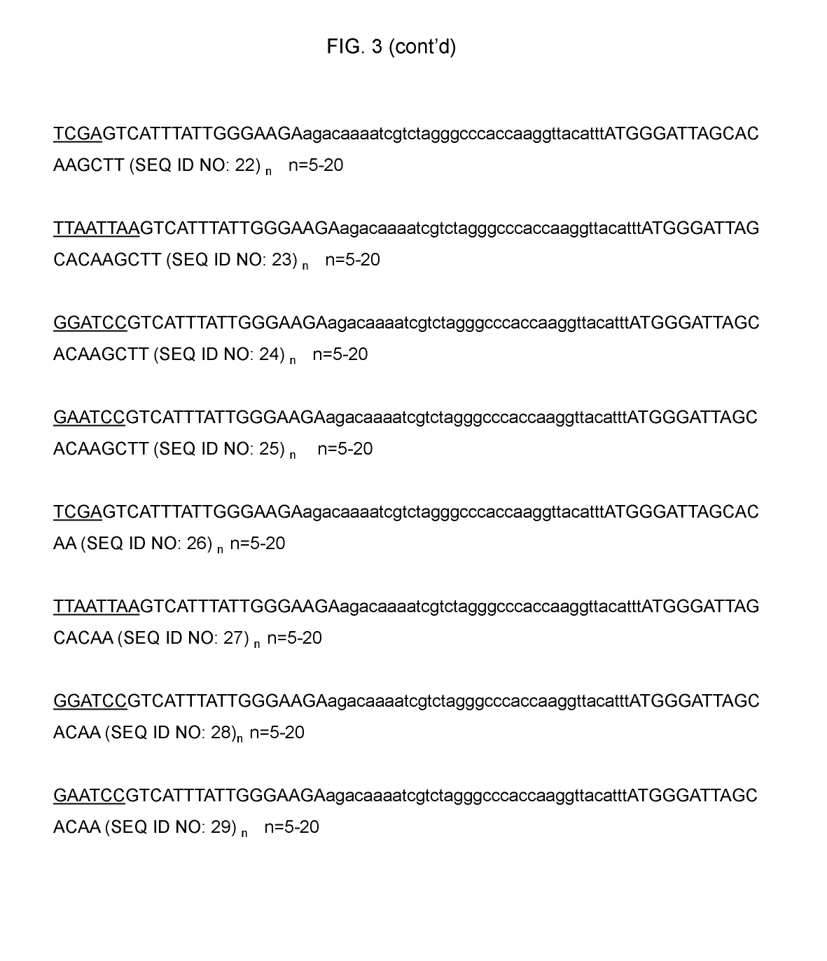

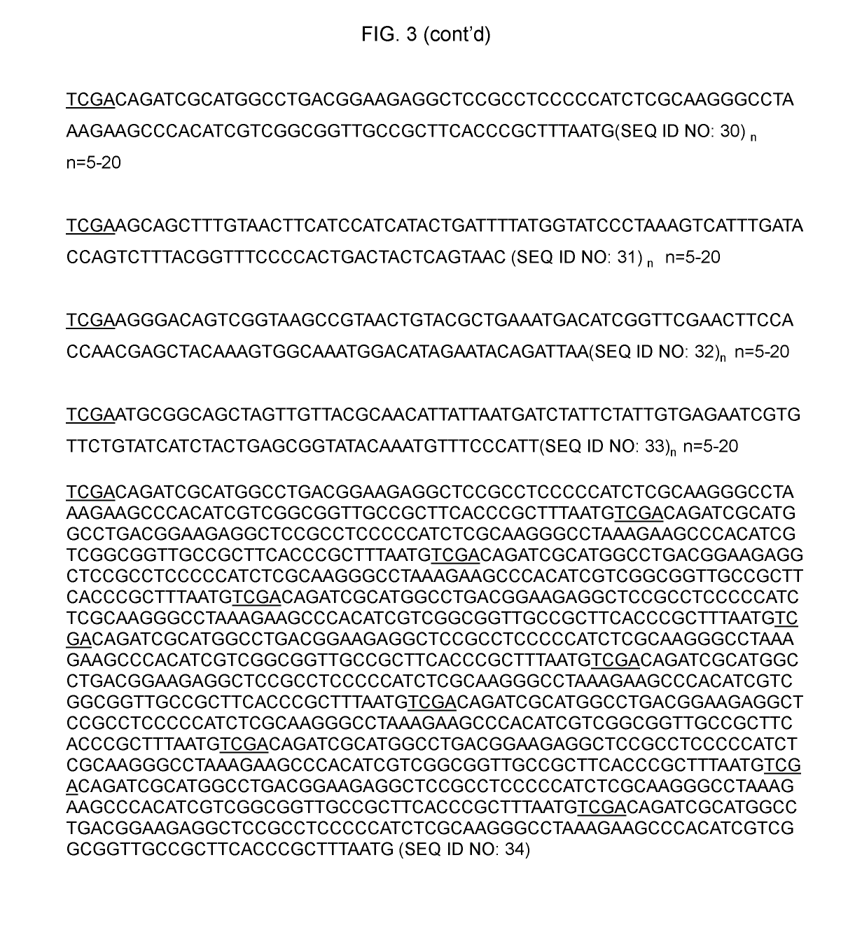

Create and validate a viral vector and digital droplet PCR (ddPCR) assay to molecularly tag and track cancer cells. A lentiviral vector has been designed that will be engineered to contain tandem, identical copies of a unique DNA sequence arranged in a head--to-tail orientation and separated by restriction enzyme recognition sites (FIG. 2). The lentiviral expression vector contains the genetic elements required for packaging, transduction, stable integration of the viral expression construct into genomic DNA, and expression of the luciferase/eGFP optical fusion reporter. High titer pseudoviral particles will be generated in producer cells, prior to transduction and eGFP flow sorting of 4T07 cells (see below). Having multiple identical molecular tag regions will increase signal to noise by “tagging” each cell with ten copies. This should permit the resolution and quantification of rare cells, which will then be tested by means of the gold standard of precisely measured mixes of molecular-tagged and non tagged cells across a 1×10⁷-fold range.

### EXAMPLE 2

Bone marrow DTCs are powerful predictors of future metastatic recurrence, and their elimination is predictive of therapeutic efficacy to prevent metastasis in breast cancer [Naume, et al., J Clin Oncol, 2014. 32(34): p. 3848-57]. Molecularly tagged syngeneic 4T07 mammary cancer cells will be implanted orthotopically into 10 “Glowing Head (GH)” Balb/c mice. Upon reaching a volume of 250mm³, primary tumors will be surgically resected (FHCRC IACUC protocol 50865). Mice will be monitored over subsequent weeks by imaging for bioluminescence to ensure that the primary tumor does not recur. Generally hundreds of quiescent DTCs are observed in bone marrow 6-weeks post-resection. Thus, at this timepoint, mice with be euthanized. All 4 femurs from each sacrificed animal will be collected. DNA will be extracted from one femoral marrow to enumerate the number of DTCs in this tissue by ddPCR. The contralateral femur will be immediately fixed and stored in OCT compound to generate femoral wholemounts. These wholemounts will be stained and imaged in the x, y and z planes to count GFP-positive DTCs per unit volume of marrow. Finally, the front femurs will be flushed into single cell suspensions, flow counted (vs. control uninoculated age-matched mice) to measure the frequency of GFP-positive cells in marrow. All three techniques will be run in triplicate on different days to assess and compare assay reproducibility.

The systems and methods disclosed herein can be used to monitor the kinetics of CTCs and ctDNA during tumorigenesis. The efficacy of targeting evolution of the dormant DTC niche as a means to chemosensitize these cells can also be measured.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically-significant reduction in the ability to quantitatively detect DTCs, CTCs, ctDNA, or RNA molecules per millimeter of whole blood.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A recombinant genetic construct comprising five to twenty repeats of a sequence selected from SEQ ID NO: 4, SEQ ID NO: 5, SEQ ID NO: 6, SEQ ID NO: 7, or SEQ ID NO: 8 with a restriction enzyme site selected from Taql, Pacl, Ascl, BamHl, Bglll, EcoRl or Xhol interspersed between each repeat.

2. A recombinant genetic construct of claim 1 comprising a repeating subunit comprising SEQ ID NO: 9, SEQ ID NO: 10, SEQ ID NO: 11, SEQ ID NO: 12, SEQ ID NO: 30, SEQ ID NO: 31, SEQ ID NO: 32, or SEQ ID NO: 33, wherein the repeating subunit is repeated five to twenty times.

3. A recombinant genetic construct of claim 1 comprising SEQ ID NO: 13 or SEQ ID NO: 34.

4. A recombinant genetic construct of claim 1 further comprising a viral vector.

5. A recombinant genetic construct of claim 4 wherein the viral vector comprises a retroviral vector.

6. A recombinant genetic construct including at least two repeats of a genetic sequence that (i) comprises 18-120 base pairs in length; (ii) is not present in the mouse genome; and (iii) is not present in the human genome; wherein the repeats in the genetic sequence are separated by a restriction enzyme site.

7. A recombinant genetic construct of claim 6 wherein the repeats of the unique genetic sequence are identical.

8. A recombinant genetic construct of claim 6 wherein each of the repeats of the unique genetic sequence can be amplified by the same primer sequence.

9. A recombinant genetic construct of claim 6 wherein each of the repeats of the unique genetic sequence share at least 95% sequence identity.

10. A recombinant genetic construct of claim 6 wherein the unique genetic sequence is artificially created, derived from an animal, or derived from a plant.

11. A recombinant genetic construct of claim 6 wherein the unique genetic sequence is derived from C. elegans or drosophila.

12. A recombinant genetic construct of claim 6 wherein the restriction enzyme site includes one or more of Taql, Pacl, Ascl, BamHl, Bglll, EcoRl or Xhol.

13. A recombinant genetic construct of claim 6 comprising a repeating subunit comprising SEQ ID NO: 9, SEQ ID NO: 10, SEQ ID NO: 11, SEQ ID NO: 12, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 SEQ ID NO: 28, SEQ ID NO: 29, SEQ ID NO: 30, SEQ ID NO: 31, SEQ ID NO: 32, or SEQ ID NO: 33, wherein the repeating subunit is repeated five to twenty times.

14. A recombinant genetic construct of claim 6 comprising SEQ ID NO: 13 or SEQ ID NO: 34.

15. A cell comprising a recombinant genetic construct of any of claims 1-14.

16. A cell of claim 15 wherein the cell is a transplanted cell.

17. A cell of claim 15 wherein the cell is a cancer cell.

18. A cell of claim 17 wherein the cancer cell is a mammalian cancer cell.

19. A cancer cell of claim 17 wherein the cancer cell is a mouse cancer cell or a human cancer cell.

20. A cancer cell of claim 17 wherein the cancer cell is an adrenal cancer cell, a bladder cancer cell, a blood cancer cell, a bone cancer cell, a brain cancer cell, a breast cancer cell, a carcinoma cell, a cervical cancer cell, a colon cancer cell, a colorectal cancer cell, a corpus uterine cancer cell, an ear, nose and throat (ENT) cancer cell, an endometrial cancer cell, an esophageal cancer cell, a gastrointestinal cancer cell, a head and neck cancer cell, a Hodgkin's disease cancer cell, an intestinal cancer cell, a kidney cancer cell, a larynx cancer cell, a leukemia cancer cell, a liver cancer cell, a lymph node cancer cell, a lymphoma cancer cell, a lung cancer cell, a melanoma cancer cell, a mesothelioma cancer cell, a myeloma cancer cell, a nasopharynx cancer cell, a neuroblastoma cancer cell, a non-Hodgkin's lymphoma cancer cell, an oral cancer cell, an ovarian cancer cell, a pancreatic cancer cell, a penile cancer cell, a pharynx cancer cell, prostate cancer cell, a rectal cancer cell, a sarcoma cancer cell, a seminoma cancer cell, a skin cancer cell, a stomach cancer cell, a teratoma cancer cell, a testicular cancer cell, a thyroid cancer cell, a uterine cancer cell, a vaginal cancer cell, a vascular tumor cancer cell, and/or a cancer cell from a metastasis thereof.

21. A method of quantitatively detecting disseminated cells in a subject comprising:
obtaining a sample derived from the subject;
detecting the presence of a unique genetic sequence and a control sequence in the sample by performing quantitative digital polymerase chain reaction (qPCR), digital PCR (dPCR) and/or next generation sequencing (NGS) on the sample; and
determining the copy number of the unique genetic sequence,
wherein the subject was administered a recombinant genetic construct of any of claims 1-14 and/or a cell comprising a recombinant genetic construct of any of claims 1-14.

22. A method of claim 21 wherein the detected disseminated cells are transplanted cells, disseminated tumor cells (DTCs), or circulating tumor cells (CTCs).

23. A method of claim 21 wherein the dPCR includes sample partition dPCR.

24. A method of claim 21 wherein the dPCR includes droplet dPCR.

25. A method of claim 21 wherein the subject is a mammal.

26. A method of claim 21 wherein the subject is a research animal.

27. A method of claim 26 wherein the research animal is a mouse, rat, monkey, pig, or dog.

28. A method of quantifying circulating tumor DNA in a subject comprising, obtaining a sample derived from the subject, detecting the presence of a unique sequence in the sample by performing quantitative digital polymerase chain reaction (qPCR), digital PCR (dPCR) and/or next generation sequencing (NGS) on the sample, and determining the copy number of the unique sequence in the sample, wherein the subject was administered a recombinant genetic construct of any of claims 1-14 or and/or a cell comprising a recombinant genetic construct of any of claims 1-14, and wherein the unique sequence is specifically associated with tumor cells in the subject, thereby quantifying circulating tumor DNA in the subject.

29. A method of claim 28 wherein the dPCR includes sample partition dPCR.

30. A method of claim 28 wherein the dPCR includes droplet dPCR.

31. A method of claim 28 wherein the subject is a mammal.

32. A method of claim 28 wherein the subject is a research animal.

33. A method of claim 32 wherein the research animal is a mouse, rat, monkey, pig, or dog.

34. A method of monitoring cancer progression or metastasis in a research subject comprising;
gene editing a cancer cell by inserting a repeated unique sequence into a human or mouse cell, wherein the unique sequence is (i) 18-120 base pairs in length, (ii) is not present in the mouse genome nor the human genome, and (iii) is repeated 5 to 20 times with restriction enzyme sites interspersed between each repeat;
administering the gene edited cancer cell to the research subject; and
monitoring the quantity of gene-edited cancer cells in the research subject by obtaining a sample derived from the subject; performing quantitative digital polymerase chain reaction (qPCR), digital PCR (dPCR) and/or next generation sequencing (NGS) on the sample; and determining the copy number of the repeated unique sequence in the sample,
thereby monitoring cancer progression or metastasis in the research subject.

35. A method of claim 34 wherein the gene-edited cancer cells are administered to induce a primary tumor in the research subject and the sample comprises a blood sample.

36. A method of evaluating a cancer treatment in a research subject comprising;
gene editing a cancer cell by inserting a repeated unique sequence into a human or mouse cell, wherein the unique sequence is (i) 18-120 base pairs in length, (ii) is not present in the mouse genome nor the human genome, and (iii) is repeated 5 to 20 times with restriction enzyme sites interspersed between each repeat;
administering the gene edited cancer cell to the research subject;
administering a cancer treatment to the subject; and
monitoring the quantity of gene-edited cancer cells in the research subject by obtaining a sample derived from the subject; performing quantitative digital polymerase chain reaction (qPCR), digital PCR (dPCR) and/or next generation sequencing (NGS) on the sample; and determining the copy number of the repeated unique sequence in the sample,
thereby evaluating the cancer treatment in the subject.

37. A method of producing a research model for cancer progression or monitoring comprising
gene editing a cancer cell by inserting a repeated unique sequence into a human or mouse cell, wherein the unique sequence is (i) 18-120 base pairs and length, (ii) is not present in the mouse genome nor the human genome, and (iii) is repeated 5 to 20 times with restriction enzyme sites between each repeat; and
inserting the gene edited cell into a research subject,
thereby producing a research model for cancer progression or monitoring.


SEQUENCE LISTING (34 sequences)

SEQ ID NO: 1 - DNA, 21 bp, C. Elegans
gagcgtcatttattgggaaga

SEQ ID NO: 2 - DNA, 23 bp, C. Elegans
aataaagcttgtgctaatcccat

SEQ ID NO: 3 - DNA, 22 bp, C. Elegans
tcgtctagggcccaccaaggtt

SEQ ID NO: 4 - DNA, 80 bp, C. elegans
gagcgtcatttattgggaagaagacaaaatcgtctagggcccaccaaggttacatttatgggattagcacaagctttatt

SEQ ID NO: 5 - DNA, 100 bp, Drosophila melanogaster
cagatcgcatggcctgacggaagaggctccgcctcccccatctcgcaagggcctaaagaagcccacatcgtcggcggttgccgcttcacccgctttaatg

SEQ ID NO: 6 - DNA, 97 bp, Golden Eagle
agcagctttgtaacttcatccatcatactgattttatggtatccctaaagtcatttgataccagtctttacggtttccccactgactactcagtaac

SEQ ID NO: 7 - DNA, 100 bp, S. pupuratus
agggacagtcggtaagccgtaactgtacgctgaaatgacatcggttcgaacttccaccaacgagctacaaagtggcaaatggacatagaatacagattaa

SEQ ID NO: 8 - DNA, 100 bp, C. intestinalis
atgcggcagctagttgttacgcaacattattaatgatctattctattgtgagaatcgtgttctgtatcatctactgagcggtatacaaatgtttcccatt

SEQ ID NO: 9 - DNA, 84 bp, Artificial sequence
  Repeated Subunit
tcgagagcgtcatttattgggaagaagacaaaatcgtctagggcccaccaaggttacatttatgggattagcacaagctttatt

SEQ ID NO: 10 - DNA, 88 bp, Artificial Sequence
  Repeated Subunit
ttaattaagagcgtcatttattgggaagaagacaaaatcgtctagggcccaccaaggttacatttatgggattagcacaagctttatt

SEQ ID NO: 11 - DNA, 86 bp, Artificial Sequence
  Repeated Subunit
ggatccgagcgtcatttattgggaagaagacaaaatcgtctagggcccaccaaggttacatttatgggattagcacaagctttatt

SEQ ID NO: 12 - DNA, 86 bp, Artificial Sequence
  Repeated Subunit
gaatccgagcgtcatttattgggaagaagacaaaatcgtctagggcccaccaaggttacatttatgggattagcacaagctttatt

SEQ ID NO: 13 - DNA, 840 bp, Artificial Sequence
  Genetic construct
tcgagagcgtcatttattgggaagaagacaaaatcgtctagggcccaccaaggttacatttatgggattagcacaagctttatttcgagagcgtcatttattgggaagaagacaaaatcgtctagggcccaccaaggttacatttatgggattagcacaagctttatttcgagagcgtcatttattgggaagaagacaaaatcgtctagggcccaccaaggttacatttatgggattagcacaagctttatttcgagagcgtcatttattgggaagaagacaaaatcgtctagggcccaccaaggttacatttatgggattagcacaagctttatttcgagagcgtcatttattgggaagaagacaaaatcgtctagggcccaccaaggttacatttatgggattagcacaagctttatttcgagagcgtcatttattgggaagaagacaaaatcgtctagggcccaccaaggttacatttatgggattagcacaagctttatttcgagagcgtcatttattgggaagaagacaaaatcgtctagggcccaccaaggttacatttatgggattagcacaagctttatttcgagagcgtcatttattgggaagaagacaaaatcgtctagggcccaccaaggttacatttatgggattagcacaagctttatttcgagagcgtcatttattgggaagaagacaaaatcgtctagggcccaccaaggttacatttatgggattagcacaagctttatttcgagagcgtcatttattgggaagaagacaaaatcgtctagggcccaccaaggttacatttatgggattagcacaagctttatt

SEQ ID NO: 14 - DNA, 79 bp, Artificial Sequence
  Repeated Subunit
tcgagtcatttattgggaagaagacaaaatcgtctagggcccaccaaggttacatttatgggattagcacaagctttat

SEQ ID NO: 15 - DNA, 84 bp, Artificial Sequence
  Repeated Subunit
ttaattaagtcatttattgggaagaagacaaaatcgtctagggcccaccaaggttacatttatgggattagcacaagctttatt

SEQ ID NO: 16 - DNA, 82 bp, Artificial Sequence
  Repeated Subunit
ggatccgtcatttattgggaagaagacaaaatcgtctagggcccaccaaggttacatttatgggattagcacaagctttatt

SEQ ID NO: 17 - DNA, 82 bp, Artificial Sequence
  Repeated Subunit
gaatccgtcatttattgggaagaagacaaaatcgtctagggcccaccaaggttacatttatgggattagcacaagctttatt

SEQ ID NO: 18 - DNA, 80 bp, Artificial Sequence
  Repeated Subunit
tcgagagcgtcatttattgggaagaagacaaaatcgtctagggcccaccaaggttacatttatgggattagcacaagctt

SEQ ID NO: 19 - DNA, 84 bp, Artificial Sequence
  Repeated Subunit
ttaattaagagcgtcatttattgggaagaagacaaaatcgtctagggcccaccaaggttacatttatgggattagcacaagctt

SEQ ID NO: 20 - DNA, 82 bp, Artificial Sequence
  Repeated Subunit
ggatccgagcgtcatttattgggaagaagacaaaatcgtctagggcccaccaaggttacatttatgggattagcacaagctt

SEQ ID NO: 21 - DNA, 82 bp, Artificial Sequence
  Repeated Subunit
gaatccgagcgtcatttattgggaagaagacaaaatcgtctagggcccaccaaggttacatttatgggattagcacaagctt

SEQ ID NO: 22 - DNA, 76 bp, Artificial Sequence
  Repeated Subunit
tcgagtcatttattgggaagaagacaaaatcgtctagggcccaccaaggttacatttatgggattagcacaagctt

SEQ ID NO: 23 - DNA, 80 bp, Artificial Sequence
  Repeated Subunit
ttaattaagtcatttattgggaagaagacaaaatcgtctagggcccaccaaggttacatttatgggattagcacaagctt

SEQ ID NO: 24 - DNA, 78 bp, Artificial Sequence
  Repeated Subunit
ggatccgtcatttattgggaagaagacaaaatcgtctagggcccaccaaggttacatttatgggattagcacaagctt

SEQ ID NO: 25 - DNA, 78 bp, Artificial Sequence
  Repeated Subunit
gaatccgtcatttattgggaagaagacaaaatcgtctagggcccaccaaggttacatttatgggattagcacaagctt

SEQ ID NO: 26 - DNA, 72 bp, Artificial Sequence
  Repeated Subunit
tcgagtcatttattgggaagaagacaaaatcgtctagggcccaccaaggttacatttatgggattagcacaa

SEQ ID NO: 27 - DNA, 76 bp, Artificial Sequence
  Repeated Subunit
ttaattaagtcatttattgggaagaagacaaaatcgtctagggcccaccaaggttacatttatgggattagcacaa

SEQ ID NO: 28 - DNA, 74 bp, Artificial Sequence
  Repeated Subunit
ggatccgtcatttattgggaagaagacaaaatcgtctagggcccaccaaggttacatttatgggattagcacaa

SEQ ID NO: 29 - DNA, 74 bp, Artificial Sequence
  Repeated Subunit
gaatccgtcatttattgggaagaagacaaaatcgtctagggcccaccaaggttacatttatgggattagcacaa

SEQ ID NO: 30 - DNA, 104 bp, Artificial Sequence
  Repeated Subunit
tcgacagatcgcatggcctgacggaagaggctccgcctcccccatctcgcaagggcctaaagaagcccacatcgtcggcggttgccgcttcacccgctttaatg

SEQ ID NO: 31 - DNA, 101 bp, Artificial Sequence
  Repeated Subunit
tcgaagcagctttgtaacttcatccatcatactgattttatggtatccctaaagtcatttgataccagtctttacggtttccccactgactactcagtaac

SEQ ID NO: 32 - DNA, 104 bp, Artificial Sequence
  Repeated Subunit
tcgaagggacagtcggtaagccgtaactgtacgctgaaatgacatcggttcgaacttccaccaacgagctacaaagtggcaaatggacatagaatacagattaa

SEQ ID NO: 33 - DNA, 104 bp, Artificial Sequence
  Repeated Subunit
tcgaatgcggcagctagttgttacgcaacattattaatgatctattctattgtgagaatcgtgttctgtatcatctactgagcggtatacaaatgtttcccatt

SEQ ID NO: 34 - DNA, 1040 bp, Artificial Sequence
  Genetic Construct
tcgacagatcgcatggcctgacggaagaggctccgcctcccccatctcgcaagggcctaaagaagcccacatcgtcggcggttgccgcttcacccgctttaatgtcgacagatcgcatggcctgacggaagaggctccgcctcccccatctcgcaagggcctaaagaagcccacatcgtcggcggttgccgcttcacccgctttaatgtcgacagatcgcatggcctgacggaagaggctccgcctcccccatctcgcaagggcctaaagaagcccacatcgtcggcggttgccgcttcacccgctttaatgtcgacagatcgcatggcctgacggaagaggctccgcctcccccatctcgcaagggcctaaagaagcccacatcgtcggcggttgccgcttcacccgctttaatgtcgacagatcgcatggcctgacggaagaggctccgcctcccccatctcgcaagggcctaaagaagcccacatcgtcggcggttgccgcttcacccgctttaatgtcgacagatcgcatggcctgacggaagaggctccgcctcccccatctcgcaagggcctaaagaagcccacatcgtcggcggttgccgcttcacccgctttaatgtcgacagatcgcatggcctgacggaagaggctccgcctcccccatctcgcaagggcctaaagaagcccacatcgtcggcggttgccgcttcacccgctttaatgtcgacagatcgcatggcctgacggaagaggctccgcctcccccatctcgcaagggcctaaagaagcccacatcgtcggcggttgccgcttcacccgctttaatgtcgacagatcgcatggcctgacggaagaggctccgcctcccccatctcgcaagggcctaaagaagcccacatcgtcggcggttgccgcttcacccgctttaatgtcgacagatcgcatggcctgacggaagaggctccgcctcccccatctcgcaagggcctaaagaagcccacatcgtcggcggttgccgcttcacccgctttaatg